(19) United States
(12) Patent Application Publication
(10) Pub. No.: US 2016/0017316 A1
(43) Pub. Date: Jan. 21, 2016

(54) ISOLATION OF MEGABASE-SIZED DNA FROM PLANT AND ANIMAL TISSUES

(71) Applicant: BioNano Genomics, Inc., San Diego, CA (US)

(72) Inventor: Michael G. Saghbini, Poway, CA (US)

(21) Appl. No.: 14/802,659

(22) Filed: Jul. 17, 2015

Related U.S. Application Data
(60) Provisional application No. 62/026,238, filed on Jul. 18, 2014.

(51) Int. Cl. C12N 15/10

(52) U.S. Cl. CPC C12N 15/1017

(57) ABSTRACT

Disclosed herein are methods for isolation of long DNA molecules, for example megabase-sized genomic DNA molecules, from a biological sample, for example plant and animal tissues.

### CROSS-REFERENCE TO RELATED APPLICATIONS

The present application claims priority under 35 U.S.C. §119(e) to U.S. Provisional Application No. 62/026,238, filed on Jul. 18, 2014, the disclosure of which is herein expressly incorporated by reference in its entirety.

### BACKGROUND

Isolation of megabase-sized DNA molecules enables genome mapping studies to decipher long range genome architecture to better understand the biology of health and disease, and in the case of plant to make more efficient crops.

To apply genome mapping studies to cancer genomics, there is a need to recover long DNA molecules, for example megabase-sized genomic DNA from animal tissues including biopsied tissue materials. Megabase-sized DNA isolation requires tissue dissociation preserving nuclear and/or cellular integrity wherein endogenous nucleases are rendered inactive. In conventional methods, the dissociated tissue is usually subjected to washing steps with isotonic conditions in the presence of EDTA, to remove some extracellular components and chelate divalent ions, prior to embedding crude cells/nuclei in agarose plugs for subsequent DNA recovery. However, tissue dissociation in aqueous solution, while preserving cellular and/or nuclear integrity, in some circumstances may not be compatible with nuclease rich tissues. Thus dissociation has traditionally been accomplished by grinding in liquid nitrogen. Equipment are available to allow processing of milligram to gram amount of tissue, i.e. FreezerMill. Besides risking DNA fragmentation due to grinding force, grinding in liquid nitrogen does not convey protection to the subsequent steps needed to extract, wash and purify cells/nuclei; solidify in agarose matrix; and achieve nuclease inactivation by complete diffusion of detergent-proteinase K mixture into the agarose matrix; a process that can take several hours.

Plant materials have the added complication of a tough cell wall that need to be fractured to release nuclei without damaging DNA, and the need to separate nuclei form unbroken cells, and cell remnants while keeping nucleases inactive which can involve lengthy density gradients. Enzymatic means of plant tissue/cell dissociation are discouraged because they occur under conditions where endogenous nucleases are active.

Furthermore, extracellular matrix and cellular contaminants (other than nucleic acid) embedded in plugs, alongside cells/nuclei, that are not protein based (i.e., glycogen, starch, cellulose, hemicellulose, pectin) or are proteinaceous but resistant to Proteinase K such as collagen, elastin, fibronectin and laminin, show up as contaminants in the recovered DNA, in standard plug lysis protocols interfering with molecular applications.

### SUMMARY

Some embodiments disclosed herein provide a method for isolating DNA from a biological sample. The method, in some embodiments, comprises (a) homogenizing the biological sample to generate a homogenate; (b) contacting the homogenate with a DNA and/or protein precipitating agent; (c) embedding the homogenate in a porous matrix; and (d) recovering DNA from the homogenate.

In some embodiments, cellular integrity, nuclear integrity, or both in the biological sample is at least partially maintained during step (a). In some embodiments, the DNA and/or protein precipitating agent comprises ethyl alcohol, methyl alcohol, isopropyl alcohol, acetone, or any combination thereof.

In some embodiments, embedding the homogenate in the porous matrix comprises dispersing the homogenate throughout the matrix. In some embodiments, the method further comprises contacting the homogenate, following step (b), with a collagenase, an elastase, a lipase, an amylase, a hyaluronidase, an RNase, a fibornectinase, a lamininase, a protease, or any combination thereof.

In some embodiments, the biological sample comprises a plant tissue and homogenizing the biological sample to generate a homogenate comprises treating the biological sample with a mechanical means, an enzymatic means, or a combination thereof. In some embodiments, the mechanical means comprises chopping the sample with a blade, macerating the sample with beads, grinding the sample with a solid material, or any combination thereof. In some embodiments, the enzymatic means comprise contacting the sample with a cellulase, a pectinase, a ligninase, a hemicellulase, or any combination thereof.

In some embodiments, the method further comprises contacting the homogenate before, after, or during step (b) with a crosslinking agent. In some embodiments, the method further comprises contacting the homogenate before, after, or during step (b) with a crosslinking agent. In some embodiments, the method further comprises separating one or more discrete DNA-containing entities from tissue fragments, intact cells, and cell remnants before step (c) and after step (b). In some embodiments, the biological sample comprises a plant tissue, an animal tissue, or both. In some embodiments, at least 30% of the DNA recovered from the homogenate is more than 20 kilobases.

In some embodiments, recovering DNA from the homogenate comprises treating the porous matrix embedded with the homogenate with one or more agents for removing non-DNA components. Non-limiting examples of the agents for removing non-DNA components include detergents, chaotropes, buffer, chelators, water soluble organic solvents, polymers, salts, acids, bases, reducing agents, or any combination thereof. In some embodiments, recovering DNA from the homogenate comprises contacting the porous matrix embedded with the homogenate with an elastase, a collagenase, hyaluronidase, an RNase, a fibornectinase, a lamininases, a lipase, a carbohydratase, a pectinase, a pectolyase, an amylase, an RNase, a hyaluronidases, or any combination thereof. In some embodiments, recovering DNA comprises melting/gelase, electroelution, or a combination thereof.

Also disclosed herein is a method for isolating DNA from a biological sample, where the method comprises: (a) contacting the biological sample with a DNA and/or protein precipitating agent; (b) homogenizing the biological sample after step (a) to generate a homogenate; (c) embedding the homogenate in a porous matrix; and (d) recovering DNA from the homogenate.

In some embodiments, the DNA and/or protein precipitating agent is ethyl alcohol, methyl alcohol, isopropyl alcohol, or acetone. In some embodiments, the method further comprises contacting the homogenate, following step (b), with a collagenase, an elastase, a lipase, an amylase, a hyaluronidase, an RNase, a fibornectinase, a lamininase, a protease, or a combination thereof. In some embodiments, the method further comprises contacting the homogenate before, after, or during step (a) with a crosslinking agent. In some embodiments, the method further comprises contacting the homogenate after, or during step (b) with a DNA and/or protein precipitating agent.

In some embodiments, the biological sample comprises a plant tissue, an animal tissue, or both. In some embodiments, at least 30% of the DNA recovered from the homogenate is more than 20 kilobases.

In some embodiments, the biological sample comprises a plant tissue and homogenizing the biological sample to generate a homogenate comprises treating the biological sample with a mechanical means, an enzymatic means, or a combination thereof. In some embodiments, the mechanical means comprises chopping the sample with a blade, macerating the sample with beads, grinding the sample with a solid material, or any combination thereof. In some embodiments, the enzymatic means comprise contacting the sample with a cellulase, a pectinase, a ligninase, a hemicellulase, or any combination thereof.

In some embodiments, the method further comprises contacting the biological sample before, after, or during step (a) with a crosslinking agent.

### BRIEF DESCRIPTION OF THE DRAWINGS

FIG. 1 is a schematic illustration of the workflow of a non-limiting embodiment of the DNA isolation methods described herein.

FIG. 2 is a pulse filed gel electrophoresis image showing DNA recovered from rat liver as described in Example 1. Yeast chromosomal markers are labeled M; the 1 Mb and 450 Kb marks are depicted. Lane 4 shows inclusion of acetic with methanol. A band present in the compression zone (marked with an arrow) indicates megabase containing DNA.

FIG. 3 is a plot showing the results of DNA recovery from various animal tissues for Irys™ mapping.

FIG. 4 is a plot showing the results of DNA recovery from rat lung tissues for Irys™ mapping.

FIGS. 5A-5B show results of rat genome de novo assembly using DNA isolated from rat liver and Irys™ mapping.

### DETAILED DESCRIPTION

In the following detailed description, reference is made to the accompanying drawings, which form a part hereof. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

The present invention describes methods for isolating long DNA molecules, for example megabase-sized genomic DNA molecules, from biological samples. The biological sample can be, or comprises, a plant tissue, an animal tissue, or both. The plant and animal tissues can be in any size, including biopsied size materials. In some embodiments, it can advantageous to stabilize nucleic and inactivate nucleases in the DNA isolation process.

In some embodiments, the DNA isolation method disclosed herein comprises: (a) homogenizing a biological sample to generate a homogenate; (b) contacting the homogenate with a DNA and/or protein precipitating agent; (c) embedding the homogenate in a solid porous matrix; and (d) recovering DNA from the homogenate.

In some embodiments, cellular integrity, nuclear integrity, or both in the biological sample is at least partially maintained during step (a). In some embodiments, it can be advantageous to substantially maintain cellular integrity, nuclear integrity, or both in the biological sample during step (a).

As used herein, the term “DNA and/or protein precipitating agent” refers to a chemical agent capable of causing precipitation of DNA, or protein, or both in a solution. DNA and/or protein precipitating agents are also known in the art as precipitant for DNA and/or proteins. Examples of DNA and/or protein precipitating agents include, but are not limited to, an acid agent, an organic solvent (including but not limited to, methanol, methanol, propanol, butanol, isopropanol, and acetone), deoxycholate, and any combination thereof. Without being bound by any particular theory, it is believed that treatment of a biological sample, for example an animal or plant tissue, with a DNA and/or protein precipitating agent can cause localized DNA precipitation inside cells and/or nuclei, or localized protein precipitation/aggregation leading to nuclease inactivity. It is also believed that the treatment with a DNA and/or protein precipitating agent may also enhance stability of cellular/nuclear structure. The time period for which the treatment of DNA and/or protein precipitating agent is carried out can vary. For example, the treatment can be carried out for few minutes to a few hours. In some embodiments, the treatment is carried out for 10 minutes to 6 hours, or 30 minutes to 3 hours. In some embodiments, the treatment is carried out overnight or for about or more than two days. The temperature under which the treatment of DNA and/or protein precipitating agent is carried out can vary. In some embodiments, it may be advantageous to carry out the treatment on ice. In some embodiments, the treatment is carried out at room temperature (i.e., between 20-25° C.) or up to 37° C.

In some embodiments, embedding the homogenate in the solid porous matrix comprises dispersing the homogenate throughout the matrix.

In some embodiments, the method further comprises contacting the homogenate, following step (b), with a collagenase, an elastase, a lipase, an amylase, a hyaluronidase, an RNase, a fibornectinase, a lamininase, a protease, or any combination thereof. In some embodiments, this enzyme treatment step is performed to remove non-DNA components and to be followed by DNA recovery by melting/gelase, electroelution, or any combination thereof.

In some embodiments, the biological sample comprises a plant tissue and homogenizing the biological sample to generate a homogenate comprises treating the biological sample with a mechanical means, an enzymatic means, or a combination thereof. In some embodiments, the mechanical means comprises chopping the sample with a blade, macerating the sample with beads, grinding the sample with a solid material, or any combination thereof. In some embodiments, the enzymatic means comprise contacting the sample with a cellulase, a pectinase, a ligninase, a hemicellulase, or any combination thereof.

The method can also comprise contacting the homogenate with a crosslinking agent. The time at which the crosslinking agent is used in the method can vary. For example, the method comprises contacting the homogenate before, after, or during step (b) with the crosslinking agent. In some embodiments, the method comprises contacting the homogenate before, after, or during step (b) with the crosslinking agent. The types of crosslinking agents that can be used in the DNA isolation methods disclosed herein are not particularly limited. For example, the crosslinking agent can be, or comprise, formaldehyde, glutaraldehydes, other aldehydes, acrolein, osmium tetroxide, or any combination thereof. In some embodiments, it may be advantageous to brief treat the homogenate with the crosslinking agent so that it is just enough to stabilize cellular and/or nuclear structures to keep the DNA compartmentalized such that the cellular and/or nuclear structures can be collected for example by centrifugation, and or filtration, for washing and embedding in porous matrix, while retaining the ability to remove contaminants other than DNA, by enzymatic and chemical means prior to DNA recovery. The time period under which the homogenate is contacted with the crosslinking agent can vary. For example, the crosslinking agent exposure time for the homogenate can be 1 minute to 2 hours, overnight, or about or longer than two days. The temperature under which the homogenate is contacted with the crosslinking agent can also vary. It may be advantageous, in some embodiments, to carry out the crosslinking agent treatment on ice. In some embodiments, the crosslinking agent treatment is carried out at room temperature (i.e., 20-25° C.) or up to 37° C.

The method can, in some embodiments, further comprises separating one or more discrete DNA-containing entities from tissue fragments, intact cells, and cell remnants. The separating step can be performed at various time during the DNA isolation process, for example, before step (c) and after step (b). Non-limiting examples of the discrete DNA-containing entity include nuclei and mitochondria.

In some embodiments, recovering DNA from the homogenate comprises treating the porous matrix embedded with the homogenate with one or more agents for removing non-DNA components. Non-limiting examples of the agents for removing non-DNA components include detergents, chaotropes, buffer, chelators, water soluble organic solvents, polymers, salts, acids, bases, reducing agents, or any combination thereof. Non-limiting examples of the polymer include polyethylene glycol, polyvinypyrrolidone, polyvinyl alcohol, ethylene glycol, or any combination thereof. In some embodiments, recovering DNA from the homogenate comprises contacting the solid porous matrix embedded with the homogenate with an elastase, a collagenase, hyaluronidase, an RNase, a fibornectinase, a lamininases, a lipase, a carbohydratase, a pectinase, a pectolyase, an amylase, an RNase, a hyaluronidases, or any combination thereof. In some embodiments, this enzyme treatment step is performed to remove non-DNA components and to be followed by the DNA recovering step. In some embodiments, recovering DNA comprises melting/gelase, electroelution, or a combination thereof.

Also disclosed herein is a method for isolating DNA from a biological sample, wherein the method comprises: (a) contacting the biological sample with a DNA and/or protein precipitating agent; (b) homogenizing the biological sample after step (a) to generate a homogenate; (c) embedding the homogenate in a solid porous matrix; and (d) recovering DNA from the homogenate. In some embodiments, the DNA and/or protein precipitating agent is an acid agent, an organic solvent (including but not limited to, methanol, methanol, propanol, butanol, isopropanol, and acetone), deoxycholate, or any combination thereof.

In some embodiments, the method further comprises contacting the homogenate, following step (b), with a collagenase, an elastase, a lipase, an amylase, a hyaluronidase, an RNase, a fibornectinase, a lamininase, a protease, or a combination thereof. In some embodiments, this enzyme treatment step is performed to remove non-DNA components and to be followed by DNA recovery by melting/gelase, electroelution, or any combination thereof.

In some embodiments, the method further comprises contacting the homogenate with a crosslinking agent. The time at which the homogenate is contacted with a crosslinking agent during the process for isolating DNA molecules can vary. For example, the homogenate can, in some embodiments, contact with the crosslinking agent before, after, or during step (a) contacting the biological sample with a DNA and/or protein precipitating agent. In some embodiments, the homogenate contacts with a DNA and/or protein precipitating agent after, or during step (b) homogenizing the biological sample after step (a) to generate the homogenate.

The DNA isolation methods disclosed herein can be used to isolate long DNA molecules, for example DNA longer than 20 Kb (kilobases), from biological samples. In some embodiments, the DNA isolated using a method disclosed herein can be, or at least, about 20 Kb, about 30 Kb, about 40 Kb, about 50 Kb, about 70 Kb, about 90 Kb, about 100 Kb, about 200 Kb, about 300 Kb, about 400 Kb, about 450 Kb, about 500 Kb, about 750 Kb, about 1 Mb (megabases), about 2 Mb, about 3 Mb, about 5 Mb, about 10 Mb, or longer. In some embodiments, the methods disclosed herein can be used to obtain a population of DNA molecules with high ratio of long DNA molecules. For example, at least about 20%, about 30%, about 40%, about 50%, about 60%, about 70%, about 80%, about 90%, about 95% of the DNA molecules isolated from the biological sample using the DNA isolation methods disclosed herein can be, or at least about 100 Kb. In some embodiments, at least about 20%, about 30%, about 40%, about 50%, about 60%, about 70%, about 80%, about 90%, about 95% of the DNA molecules isolated from the biological sample using the DNA isolation methods disclosed herein can be, or at least about 20 Kb. In some embodiments, at least about 20%, about 30%, about 40%, about 50%, about 60%, about 70%, about 80%, about 90%, about 95% of the DNA molecules isolated from the biological sample using the DNA isolation methods disclosed herein can be, or at least about 450 Kb. In some embodiments, at least about 20%, about 30%, about 40%, about 50%, about 60%, about 70%, about 80%, about 90%, about 95% of the DNA molecules isolated from the biological sample using the DNA isolation methods disclosed herein can be, or at least, about 1 Mb, about 2 Mb, about 3 Mb, about 5 Mb, about 10 Mb, or longer. In some embodiments, about 20%, about 30%, about 40%, about 50%, about 60%, about 70%, about 80%, about 90%, about 95% of the DNA molecules isolated from the biological sample using the DNA isolation methods disclosed herein can be, or at least, about 20 Kb, about 30 Kb, about 40 Kb, about 50 Kb, about 70 Kb, about 90 Kb, about 100 Kb, about 200 Kb, about 300 Kb, about 400 Kb, about 450 Kb, about 500 Kb, about 750 Kb, about 1 Mb, about 2 Mb, about 3 Mb, about 5 Mb, about 10 Mb, or longer.

The source of the biological sample that is suitable to use in the DNA isolation methods disclosed herein is not particularly limited. For example, the biological sample can be originated from a plant or an animal, for example a biological sample comprising a plant issue, an animal tissue, or both. In some embodiments, the biological sample is a clinical sample originated from a human, for example a biopsy sample. The biological sample can be or comprise, in some embodiments, an animal tissue, for example heart tissue, liver tissue, lung tissue, brain tissue, kidney tissue, prostate tissue, uterine tissue, colon tissue, head & neck tissue, pancreatic tissue, muscle tissue, breast tissue, stomach tissue, ovary tissue, skin tissue, connective tissue, blood, tissue that contains body fluids or contains trace of such fluids (e.g., blood, CSF, urine, saliva, mammary fluid), or any combination thereof. The animal tissue may be a normal tissue or a diseased or injury tissue, such as cancerous, inflamed, infected, congenitally diseased, functional comprised (e.g., diabetes, neurodegenerative, or atrophy), traumatized or environmentally insulted. In some embodiments, the biological sample can be, or comprises, a plant tissue, for example, tissue originated from leaves, roots, flowers, fruits, stems, seeds, or any combination thereof. In some embodiments, the tissue can be, or comprises, epidermis tissue, parenchyma tissue, meristematic tissue, sclerenchyma tissue, xylem tissue, phloem tissue, or any combination thereof. The plant tissue may be a normal tissue or a diseased or injury tissue, such as a tissue that is infected, congenitally diseased, traumatized or environmentally insulted. The tissue can either be unmodified or processed before being subject to the method disclosed herein for isolating DNA.

One of ordinary skill in the art can appreciate that the DNA isolation methods disclosed herein can be applied to a complex biological sample in which contaminating molecules are present, or to a sample comprising a multiplicity of cells.

In some embodiments, the methods described herein enable processing biopsy-sized tissue without grinding in liquid nitrogen. As described herein, various tissues can be successfully processed, for example rat liver, rat brain, rat kidney, rat lung, and mouse prostate. Without being bound by any particular theory, it is believed that some embodiments of the method described herein can stabilize DNA in tissues and/or tissue homogenates by the treatment of a DNA and/or protein precipitating agent to precipitate the DNA inside cells and/or nuclei. In some embodiments, the methods protect against shearing and nucleases due to a generalized protein precipitation process brought about by the treatment of the DNA and/or protein precipitating agent (e.g., alcohol).

Non-Limiting Exemplary Processes for Isolating DNA from Biological Samples

Provided herein are various non-limiting examples of methods/processes disclosed herein for isolating DNA molecules from biological samples.

Processes (A): in some embodiments, a biological sample, such as an animal tissue, can be homogenized in an isotonic buffer to generate a homogenate. Without being bound to any particular theory, it is believe that homogenization the sample in the isotonic buffer can help to preserve cellular and/or nuclear integrity in the sample. The resulting homogenate can be treated (e.g., contacted) with one or more DNA and/or protein precipitating agents. Examples of the DNA and/or protein precipitating agent include, but are not limited to, ethyl alcohol (also known as ethanol), methyl alcohol (also known as methanol), isopropyl alcohol (also known as isopropanol), acetone, and any combination thereof. In some embodiments, The homogenate can be embedded in a porous matrix, for example the homogenate can be dispersed throughout the matrix, for subsequent DNA recovery.

In some embodiments, the animal tissue can be homogenized in an isotonic buffer to generate a homogenate. The resulting homogenate is treated (e.g., contacted) with a DNA and/or protein precipitating agent, such as ethyl alcohol, methyl alcohol, isopropyl alcohol, acetone, or a combination thereof. The homogenate can be further treated with a collagenase, an elastase, a lipase, an amylase, a hyaluronidase, an RNase, a fibornectinase, a lamininase, a protease, or a combination thereof to, for example, remove animal ExtraCellular Matrix (ECM) components, and optionally cytosolic components other than nucleic acid. The homogenate is embedded in a porous matrix, for example the homogenate is dispersed throughout the matrix, for subsequent DNA recovery.

Processes (B): in some embodiments, a biological sample, such as an animal tissue, can be homogenized in an isotonic buffer to produce a homogenate. As described herein, it can be advantageous, in some embodiments, to preserve cellular and/or nuclear integrity in the tissue during the homogenization process. The resulting homogenate can be treated (e.g., contacted) with a cross linking agent to stabilize cellular and/or nuclear integrity. The homogenate can be further treated with a DNA and/or protein precipitating agent, for example ethyl alcohol, methyl alcohol, isopropyl alcohol, acetone, or a combination thereof. The homogenate can be further treated with a collagenase, an elastase, a lipase, an amylase, a hyaluronidase, an RNase, a fibornectinase, a lamininase, a protease, or a combination thereof. The homogenate can be embedded in a porous matrix, for example the homogenate can be dispersed throughout the matrix, for subsequent DNA recovery. In some embodiments, the treatment with the DNA and/or protein precipitating agent is not performed. In some embodiments, the treatment with collagenase, elastase, lipase, amylase, hyaluronidase, RNase, fibornectinase, lamininase, and protease is also not performed. In some embodiments, both of the treatment with the DNA and/or protein precipitating agent and treatment with collagenase, elastase, lipase, amylase, hyaluronidase, RNase, fibornectinase, lamininase, and protease are not performed.

Processes (C): in some embodiments, a biological sample, such as an animal tissue, is treated (e.g., contacted) with a DNA and/or protein precipitating agent, such as ethyl alcohol, methyl alcohol, isopropyl alcohol, acetone, or a combination thereof. The tissue can be treated with a collagenase, an elastase, a lipase, an amylase, a hyaluronidase, an RNase, a fibornectinase, a lamininase, a protease, or a combination thereof. The tissue can be homogenized in an isotonic buffer to maintain that cellular/nuclear integrity to generate a homogenate. The homogenate can then be embedded in a porous matrix, for example the homogenate can be dispersed throughout the matrix, for subsequent DNA recovery. In some embodiments, the treatment with collagenase, elastase, lipase, amylase, hyaluronidase, RNase, fibornectinase, lamininase, and protease is not performed.

Processes (D): in some embodiments, a biological sample, such as an animal tissue, is treated (e.g., contacted) with a crosslinking agent to stabilize cellular and/or nuclear integrity. In some embodiments, the tissue is then washed to remove crosslinking agent. The tissue can be treated (e.g., contacted) with a DNA and/or protein precipitating agent, such as ethyl alcohol, methyl alcohol, isopropyl alcohol, acetone, or a combination thereof. The tissue can be further treated with a collagenase, an elastase, a lipase, an amylase, a hyaluronidase, an RNase, a fibornectinase, a lamininase, a protease, or a combination thereof. The tissue can be homogenized in an isotonic buffer to generate a homogenate. The homogenate can be embedded in a porous matrix, for example the homogenate can be dispersed throughout the matrix, for subsequent DNA recovery. In some embodiments, either one or both of the treatment with the DNA and/or protein precipitating agent, and the treatment with collagenase, elastase, lipase, amylase, hyaluronidase, RNase, fibornectinase, lamininase, and protease, is not performed.

Processes (E): in some embodiments, a biological sample, such as an animal tissue, is treated (e.g., contacted) with a crosslinking agent, for example to stabilize cellular and/or nuclear integrity. In some embodiments, the tissue can then be washed to remove crosslinking agent. The tissue is treated (e.g., contacted) with ethyl alcohol, methyl alcohol, isopropyl alcohol, acetone, a DNA and/or protein precipitating agent, or a combination thereof. The tissue can be homogenized in an isotonic buffer to generate a homogenate. The homogenate can then be treated with a collagenase, an elastase, a lipase, an amylase, a hyaluronidase, an RNase, a fibornectinase, a lamininase, a protease, or a combination thereof. The homogenate can be embedded in a porous matrix, for example the homogenate is dispersed throughout the matrix, for subsequent DNA recovery. In some embodiments, either one or both of the treatment with the crosslinking agent and the treatment with the DNA/protein precipitating agent, is not performed.

Processes (F): in some embodiments, a biological sample, such as an animal tissue, is treated (e.g., contacted) with a crosslinking agent, for example to stabilize cellular and/or nuclear integrity. In some embodiments, the tissue can then be washed to remove crosslinking agent. The tissue can be treated (e.g., contacted) with a DNA and/or protein precipitating agent, such as ethyl alcohol, methyl alcohol, isopropyl alcohol, acetone, or a combination thereof. The tissue can be treated with a collagenase, an elastase, a lipase, an amylase, a hyaluronidase, an RNase, a fibornectinase, a lamininase, a protease that preserves cellular integrity, or a combination thereof to disintegrate tissue. The disintegrated tissue can be embedded in a porous matrix, for example the disintegrate tissue can be dispersed throughout the matrix, for subsequent DNA recovery. In some embodiments, either one or both of the treatment with the crosslinking agent and the treatment with the DNA/protein precipitating agent, is not performed.

Processes (G): in some embodiments, a biological sample, such as an animal tissue, is treated (e.g., contacted) with a crosslinking agent, for example to stabilize cellular and/or nuclear integrity. In some embodiments, the tissue can then be washed to remove crosslinking agent. The tissue can be homogenized in an isotonic buffer to generate a homogenate. The homogenate can then be treated with a DNA and/or protein precipitating agent, such as ethyl alcohol, methyl alcohol, isopropyl alcohol, acetone, or a combination thereof. The homogenate can be further treated with a collagenase, an elastase, a lipase, an amylase, a hyaluronidase, an RNase, a fibornectinase, a lamininase, a protease, or a combination thereof. The homogenate can be embedded in a porous matrix, for example the homogenate can be dispersed throughout the matrix, for subsequent DNA recovery. In some embodiments, the treatment with collagenase, elastase, lipase, amylase, hyaluronidase, RNase, fibornectinase, lamininase, and protease, is not performed.

Processes (H): in some embodiments, a biological sample, such as an animal tissue, is treated (e.g., contacted) with a DNA and/or protein precipitating agent, such as ethyl alcohol, methyl alcohol, isopropyl alcohol, acetone, or a combination thereof. The tissue can be homogenized in an isotonic buffer to generate a homogenate. In some embodiments, the homogenate can then be treated with a crosslinking agent, for example to stabilize cellular and/or nuclear integrity. In some embodiments, the homogenate can be washed to remove crosslinking agent. The homogenate can be further treated with a collagenase, an elastase, a lipase, an amylase, a hyaluronidase, an RNase, a fibornectinase, a lamininase, a protease, or a combination thereof. The homogenate can be embedded in a porous matrix, for example the homogenate can be dispersed throughout the matrix, for subsequent DNA recovery. In some embodiments, the treatment with collagenase, elastase, lipase, amylase, hyaluronidase, RNase, fibornectinase, lamininase, and protease, is not performed. In some embodiments, the homogenization step is not performed.

Processes (K): in some embodiments, a biological sample, such as a plant tissue, is disintegrated, for example by grinding in liquid nitrogen or chopping with a blade. The resulting disintegrated material can be treated (e.g., contacted) with a DNA and/or protein precipitating agent, such as ethyl alcohol, methyl alcohol, isopropyl alcohol, acetone, or a combination thereof. Nuclei and/or discrete entities comprising DNA can be separated from tissue fragments, intact cells, and cell remnants. Separated nuclei and/or discrete entities can be embedded in a porous matrix, for example they can be dispersed throughout the matrix, for subsequent DNA recovery.

Processes (K): in some embodiments, a biological sample, such as a plant tissue, is disintegrated, for example by grinding in liquid nitrogen or chopping with a blade. The resulting disintegrated material is treated (e.g., contacted) with a DNA and/or protein precipitating agent, such as ethyl alcohol, methyl alcohol, isopropyl alcohol, acetone, or a combination thereof. The disintegrated material can be further treated (e.g., contacted) with cellulase, pectinase, ligninase, hemicellulase, or any combination thereof. Nuclei and/or discrete entities comprising DNA can be separated from tissue fragments, intact cells, and cell remnants. Separated nuclei and/or discrete entities can be embedded in a porous matrix, for example they can be dispersed throughout the matrix, for subsequent DNA recovery.

Processes (L): in some embodiment, a biological sample, such as a plant tissue, is disintegrated, for example by grinding in liquid nitrogen or chopping with a blade. The resulting disintegrated material is treated (e.g., contacted) with a cross linking agent to stabilize cellular and/or nuclear integrity. The disintegrated material can be further treated with a DNA and/or protein precipitating agent, such as ethyl alcohol, methyl alcohol, isopropyl alcohol, acetone, or a combination thereof. The disintegrated material can be further treated with cellulase, pectinase, ligninase, hemicellulase, or any combination thereof. Nuclei and/or discrete entities comprising DNA can be separated from tissue fragments, intact cells, and cell remnants. Separated nuclei and/or discrete entities can be embedded in a porous matrix, for example they can be dispersed throughout the matrix, for subsequent DNA recovery. In some embodiment, the treatment with the DNA and/or protein precipitating agent is not performed. In some embodiments, the treatment with cellulase, pectinase, ligninase, and hemicellulase is not performed. In some embodiments, both the treatment with the DNA and/or protein precipitating agent and the treatment with cellulase, pectinase, ligninase, and hemicellulase are not performed.

Processes (M): in some embodiments, a biological sample, such as a plant tissue, is treated (e.g., contacted) with a crosslinking agent, for example to stabilize cellular and/or nuclear integrity. In some embodiments, the tissue can then be washed to remove the crosslinking agent. The tissue can be treated (e.g., contacted) with a DNA and/or protein precipitating agent, such as ethyl alcohol, methyl alcohol, isopropyl alcohol, acetone, or a combination thereof. The tissue can be further treated with a cellulase, pectinase, ligninase, hemicellulase, or any combination thereof. The tissue can be homogenized in an isotonic buffer generate a homogenate. Nuclei and/or discrete entities comprising DNA can be separated from tissue fragments, intact cells, and cell remnants. Separated nuclei and/or discrete entities can be embedded in a porous matrix, for example they can be dispersed throughout the matrix, for subsequent DNA recovery. In some embodiments, the treatment with the crosslinking agent is not performed. In some embodiments, the treatment with the DNA and/or protein precipitating agent is not performed.

Processes (N): in some embodiments, a biological sample, such as a plant tissue, is treated (e.g., contacted) with a crosslinking agent, for example to stabilize cellular and/or nuclear integrity. In some embodiments, the tissue can then be washed to remove the crosslinking agent. The tissue can be treated (e.g., contacted) with a DNA and/or protein precipitating agent, such as ethyl alcohol, methyl alcohol, isopropyl alcohol, acetone, or a combination thereof. The tissue can be disintegrated, for example by grinding in liquid nitrogen or chopping with a blade. The disintegrated tissue can be further treated with a cellulase, pectinase, ligninase, hemicellulase, or any combination thereof. Nuclei and/or discrete entities comprising DNA can be separated from tissue fragments, intact cells, and cell remnants. Separated nuclei and/or discrete entities can be embedded in a porous matrix, for example they can be dispersed throughout the matrix, for subsequent DNA recovery. In some embodiments, the treatment with the crosslinking agent is not performed. In some embodiments, the treatment with the protein precipitating agent is not performed.

Processes (O): in other embodiments, a biological sample, such as a plant tissue, is treated (e.g., contacted) with a crosslinking agent, for example to stabilize cellular and/or nuclear integrity. In some embodiments, the tissue can then be washed to remove the crosslinking agent. The tissue can be treated (contacted) with a DNA and/or protein precipitating agent, such as ethyl alcohol, methyl alcohol, isopropyl alcohol, acetone, or a combination thereof. The tissue can be treated with a cellulase, pectinase, ligninase, hemicellulase, or any combination thereof. Nuclei and/or discrete entities comprising DNA can be separated from tissue fragments, intact cells, and cell remnants. Separated nuclei and/or discrete entities can be embedded in a porous matrix, for example they can be dispersed throughout the matrix, for subsequent DNA recovery. In some embodiments, the treatment with the crosslinking agent is not performed. In some embodiments, the treatment with the DNA/protein precipitating agent is not performed.

Processes (P): in some embodiments, a biological sample, such as a plant tissue, is treated (e.g., contacted) with a crosslinking agent, for example to stabilize cellular and/or nuclear integrity. In some embodiments, the tissue can be then washed to remove the crosslinking agent. The tissue can be disintegrated, for example by grinding in liquid nitrogen or chopping with a blade. The disintegrated tissue can be treated (e.g., contacted) with a DNA and/or protein precipitating agent, such as ethyl alcohol, methyl alcohol, isopropyl alcohol, acetone, or a combination thereof. The disintegrated tissue can be further treated with a cellulase, pectinase, ligninase, hemicellulase, or any combination thereof. Nuclei and/or discrete entities comprising DNA can be separated from tissue fragments, intact cells, and cell remnants. Separated nuclei and/or discrete entities can be embedded in a porous matrix, for example they can be dispersed throughout the matrix, for subsequent DNA recovery. In some embodiments, the treatment with cellulase, pectinase, ligninase, and hemicellulase is not preformed.

Processes (Q): in some embodiments, a biological sample, such as a plant tissue, is treated (e.g., contacted) with a DNA and/or protein precipitating agent, such as ethyl alcohol, methyl alcohol, isopropyl alcohol, acetone, or a combination. The tissue can be disintegrated, for example by grinding in liquid nitrogen or chopping with a blade. The disintegrated tissue can be treated (e.g., contacted) with a crosslinking agent, for example to stabilize cellular and/or nuclear integrity. The disintegrated tissue can then be washed to remove the crosslinking agent. The disintegrated tissue can be further treated with a cellulase, pectinase, ligninase, hemicellulase, or any combination thereof. Nuclei and/or discrete entities comprising DNA can be separated from tissue fragments, intact cells, and cell remnants. Separated nuclei and/or discrete entities can be embedded in a porous matrix, for example they can be dispersed throughout the matrix, for subsequent DNA recovery. In some embodiments, the treatment with cellulase, pectinase, ligninase, and hemicellulase is not performed.

Table 1 provides a brief summary of the processing workflow for the non-limiting DNA isolation processes (A)-(Q) as discussed above. Each of processes (A)-(Q) can be used in isolating DNA molecules from biological samples. In some embodiments, it can be advantageous to use processes (A)-(H) to isolate DNA molecules from biological samples comprising animal tissues. In some embodiments, it can be advantageous to use processes (A)-(H) to isolate DNA molecules from biological samples comprising animal tissues. In some embodiments, the step of treatment with the crosslinking agent (i.e. “crosslink”) and the step of treatment with the DNA and/or protein precipitating agent can be carried out in any order or simultaneously. In Table 1, the term “collagenase-like enzyme” refers to an enzyme or an enzyme mixture such as collagenase, elastase, lipase, amylase, hyaluronidase, RNase, fibornectinase, lamininase, protease, or a combination thereof; the term “cellulase-like enzyme” refers to an enzyme or an enzyme mixture such as cellulase, pectinase, ligninase, hemicellulose, or a combination thereof; and the term “Separate” refers to an action such as low speed spins and density gradients to purify nuclei and/or entities comprising DNA away from tissue fragment, intact cells, and cell remnants.

TABLE 1
Brief summary of the processing workflow for DNA isolation processes (A)-(Q)

| | Processes | | | | | | | | | | | | | |
|---|---|---|---|---|---|---|---|---|---|---|---|---|---|---|
| | A | B | | C | D | | E | | | F | | | G | H |
| Tissue | ✓ | ✓ | ✓ | ✓ | ✓ | ✓ | ✓ | ✓ | ✓ | ✓ | ✓ | ✓ | ✓ | ✓ |
| Crosslink | | | | | ✓ | ✓ | | ✓ | ✓ | | ✓ | ✓ | ✓ | |
| Treat with DNA and | | | | ✓ | | ✓ | ✓ | | ✓ | ✓ | | ✓ | | ✓ |
| Protein precipitating | | | | | | | | | | | | | | |
| agent | | | | | | | | | | | | | | |
| Treatment with | | | | ± | ± | ± | | | | | | | | |
| collagenase-like enzyme | | | | | | | | | | | | | | |
| Homogenize | ✓ | ✓ | ✓ | ✓ | ✓ | ✓ | ✓ | ✓ | ✓ | | | | ✓ | ✓ |
| Crosslink | | ✓ | ✓ | | | | | | | | | | | ✓ |
| Treat with DNA and | ✓ | | ✓ | | | | | | | | | | ✓ | |
| Protein precipitating | | | | | | | | | | | | | | |
| agent | | | | | | | | | | | | | | |
| Treatment with | ± | ± | ± | | | | ✓ | ✓ | ✓ | ✓ | ✓ | ✓ | ± | ± |
| collagenase-like enzyme | | | | | | | | | | | | | | |
| Embed in porous matrix | ✓ | ✓ | ✓ | ✓ | ✓ | ✓ | ✓ | ✓ | ✓ | ✓ | ✓ | ✓ | ✓ | ✓ |
| Recover DNA | ✓ | ✓ | ✓ | ✓ | ✓ | ✓ | ✓ | ✓ | ✓ | ✓ | ✓ | ✓ | ✓ | ✓ |
| | Processes | | | | | | | | | | | | | |
| | K | L | | M | | | N | | | O | | | P | Q |
| Tissue | ✓ | ✓ | ✓ | ✓ | ✓ | ✓ | ✓ | ✓ | ✓ | ✓ | ✓ | ✓ | ✓ | ✓ |
| Crosslink | | | | | ✓ | ✓ | | ✓ | ✓ | | ✓ | ✓ | ✓ | |
| Treat with DNA and | | | | ✓ | | ✓ | ✓ | | ✓ | ✓ | | ✓ | | ✓ |
| Protein precipitating | | | | | | | | | | | | | | |
| agent | | | | | | | | | | | | | | |
| Treat with cellulase-like | | | | ✓ | ✓ | ✓ | | | | | | | | |
| enzyme | | | | | | | | | | | | | | |
| Disintegrate | ✓ | ✓ | ✓ | | | | ✓ | ✓ | ✓ | | | | ✓ | ✓ |
| Homogenize | | | | ✓ | ✓ | ✓ | | | | | | | | |
| Crosslink | | ✓ | ✓ | | | | | | | | | | | ✓ |
| Treat with DNA and | ✓ | | ✓ | | | | | | | | | | ✓ | |
| Protein precipitating | | | | | | | | | | | | | | |
| agent | | | | | | | | | | | | | | |
| Treat with cellulase-like | ± | ± | ± | | | | ✓ | ✓ | ✓ | ✓ | ✓ | ✓ | ± | ± |
| enzyme | | | | | | | | | | | | | | |
| Separate | ✓ | ✓ | ✓ | ✓ | ✓ | ✓ | ✓ | ✓ | ✓ | ✓ | ✓ | ✓ | ✓ | ✓ |
| Embed in porous matrix | ✓ | ✓ | ✓ | ✓ | ✓ | ✓ | ✓ | ✓ | ✓ | ✓ | ✓ | ✓ | ✓ | ✓ |
| Recover DNA | ✓ | ✓ | ✓ | ✓ | ✓ | ✓ | ✓ | ✓ | ✓ | ✓ | ✓ | ✓ | ✓ | ✓ |

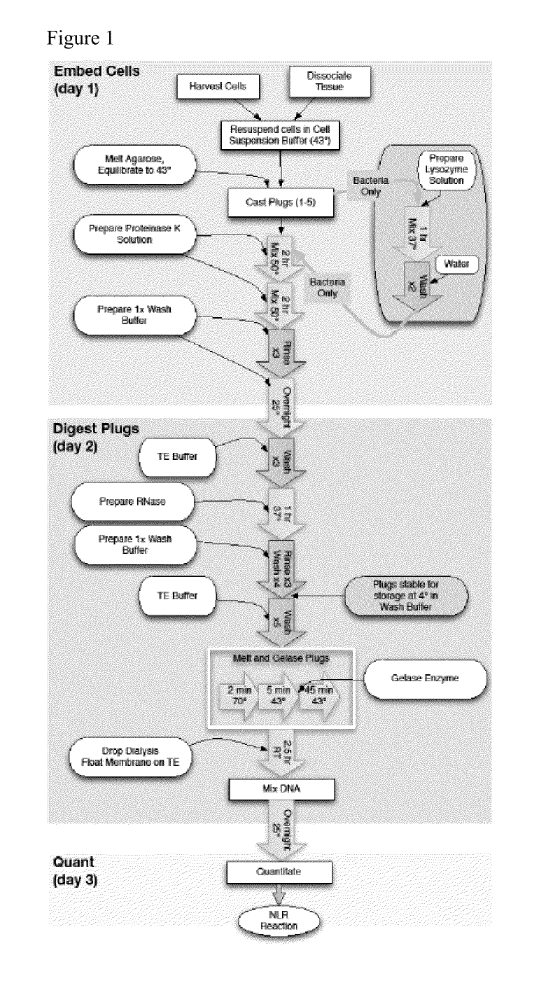

FIG. 1 provides a schematic illustration of the workflow of a non-limiting embodiment of the DNA isolation methods described herein.

As used herein, the expressions “disintegration of tissues” and “homogenization of tissues” are used interchangeably. The tissues can be, for example, animal tissues or plant tissues. Many methods are known for disintegrating or homogenizing animal and plants tissues. For example, animal tissue homogenization can be achieved using a dounce type homogenizer (e.g., Tenbroeck, Potter-Elvehjem, Dounce) available in different sizes preferably with frosting (on mortar and pestle) to better disintegrate tough tissues. Soft tissue can also be disintegrated by pushing through a cell dissociation sieve having a mesh size opening ranging for 5 micron to 500 micron (e.g., Sigma cat numbers: S0770, S0895, S1020, S3770, S3895, S4020, S4145). Tissue disintegration can also be done with a rotating blade such as TissueRuptor by Qiagen (cat #9001271), and ULTRA TURRAX Tube Disperser Workstation (Sigma cat # Z722332). Tissue disintegration can also be achieved by grinding in liquid nitrogen. In some embodiments, it can be advantageous to disintegrate animal tissue biopsies in dounce type homogenizers as included in Wheaton 358204 micro tissue grinder kit.

In some embodiments, throughout processing of biological samples, for example plant and animal tissues, until even dispersion in the porous matrix, genomic DNA remains compartmentalized in cells, nuclei and or entities that can be collected by centrifugation, and/or filtration enabling washing and gradient purification while allowing for degradation and removal of contaminants other than nucleic acid by enzymatic or chemical means when cells, nuclei and or entities comprising DNA are embedded in porous matrix.

In the methods disclosed herein, the treatment step with the cross linking agent, and the treatment step with the DNA/protein precipitating agent can be in any order. In some embodiments, the both steps can occur simultaneously.

In some embodiments, collagenases, elastases, fibornectinases, and lamininases along with protease used for tissue dissociation to produce cells, such as clostripain, trypsin and dispase; hyaloronidases; and carbohydratases, in any combination can be used to degrade animal extracellular matrix (ECM) material that is refractory to standard proteinase K digest. Crude enzymes are available that comprises many of these different activities at different level, i.e. Sigma catalog numbers: C0130, C1639 and C9891. Cellulases, pectinases, ligninases, and hemicellulases are also available as crude enzyme mixtures comprising various activities for use to degrade plant extracellular matrix.

As disclosed herein, nuclei and/or discrete entities comprising DNA can be separated from plant or animal tissue fragments, intact cells, and cell remnants by gravity settling, low speed centrifugation such that nuclei are not pelleted, and density gradient centrifugation involving: percoll, ficoll, sucrose, and other density forming material, in any combination.

According to some embodiments, a porous matrix is provided. The porous matrix can comprise pores to permit the movement of molecules such as polynucleotide molecules (e.g. DNA molecules being removed from the homogenate) in, out, and within the matrix. In some embodiments, a porous matrix is formed from a precursor material. For example, a liquid agarose solution can form a matrix upon cooling. Accordingly, in some embodiments, a homogenate is embedded in a porous matrix by contacting the homogenate with the precursor material, and then forming the matrix so that the homogenate is embedded therein.

The porous matrix can be, for example a solid porous matrix. In some embodiments, the porous matrix comprises a synthetic polymer, a naturally-occurring polymer, or a combination of the two. Various materials can be used to make the porous matrix. For example, the porous matrix can be made of, or comprise, agarose. As other examples, porous matrix can be made of, or comprise, polyacrylamide, gelatin, collagen, fibrin, chitosan, alginate, hyaluronic acid, or any combination thereof. Porous matrix can be in various forms or shapes, for example, a plug, a microbead, a microlayer, or any shape. In some embodiments, the porous matrix is, or comprises, an agarose matrix, a polyacrylamide matrix, a gelatin matrix, a collagen matrix, a fibrin matrix, a chitosan matrix, an alginate matrix, a hyaluronic acid matrix, or a combination of two or more of the listed items, for example two, three, four, five, six, seven, or eight of the listed items. In some embodiments, a combination of precursors of two or more of the listed materials is combined, and formed into a porous matrix. In some embodiments, porous matrices formed of two or more of the listed materials are formed and then combined. In some embodiments, the porous matrix is an agarose matrix. In some embodiments, the porous matrix is a polysaccharide-based matrix. As some samples, for example nucleic acids, can be soluble in aqueous environments, in some embodiments, the porous matrix comprises an aqueous environment. In some embodiments, the matrix itself is disposed in an aqueous environment, for example an aqueous buffer.

It can be useful for a porous matrix to include one or more functional groups, depending on the desired function of the porous matrix. For example, without being limited by any particular theory, removal of hydrophobic materials from the matrix can be facilitated by the inclusion of hydrophilic functional groups in the matrix. For example, without being limited by any particular theory, immobilization of polynucleotides in the matrix can be facilitated by positively charged functional groups in the matrix. As such, in some embodiments, the porous matrix comprises a silane, a positively charged group, a negatively charged group, a zwitterionic group, a polar group, a hydrophilic group, a hydrophobic group, or a combination of two or more of the listed items, for example two, three, four, five, six, or seven of the listed items. Non-limiting examples of suitable porous matrix are describe in PCT/US2015/019027 filed on Mar. 5, 2015, the content of which is hereby incorporated by reference in its entirety.

As disclosed herein, in some embodiments, DNA recovery comprises treatment of the porous matrix comprising the homogenate (i.e., crude DNA-containing material) with detergent-proteinase K mixture followed by extensive washing prior to recovering the DNA by melting and/or gelase. Insoluble matter carried over with cell/nuclei preparation when embedded into the porous matrix can contaminate the DNA in some embodiments if such matter is not degraded by proteinase K, the standard treatment for cleaning plugs. Some major components of plant and animal extracellular matrix (ECM), for example cellulose, hemicelluloses, pectin, hyaluronic acid, glycosaminoglycan, collagen, elastin, fibronectin and laminin, can be refractory to proteinase K digestion.

Some enzymes that degrade animal and plant extracellular matrix may require divalent cations, which serve as cofactors for nucleases if not inactivated. In some embodiments, prior treatment with one or more crosslinking agents, such as acetone and the alcohols: ethyl alcohol, methyl alcohol, isopropyl alcohol, can stabilize DNA and inactivate nucleases.

In some embodiments, DNA recovery comprises treating the porous matrix comprising the crude DNA with ECM degrading enzymes before or after proteinase K treatment. In cases where degrading enzymes are used after proteinase K, an additional proteinase K treatment may be performed. As ECM degrading enzymes tend to be a mixture of different enzymes that attack different components of the ECM and has the potential to include nuclease, treatment is carried out under the conditions where DNA is protected. In some embodiments, the DNA is protected by precipitation.

Chemical means can also be employed to clean ECM and cytosolic contaminants trapped in the porous matrix. Examples of chemical means include, but are not limited by, a detergent, a chaotrope, a buffer, a chelator, a water soluble organic solvent, a polymer (e.g., polyethylene glycol, polyvinypyrrolidone, polyvinyl alcohol, ethylene glycol), a salt, an acid, a base, a reducing agent, or a combination thereof.

Non-limiting examples of organic solvent rendered miscible with water include: chlorofrom/methanol (MeOH) solution 1: MeOH: 50%; Chloroform: 33%; H₂O: 16.6%; chlorofrom/methanol (MeOH) solution 2: MeOH: 43.75%; Chloroform: 50%; H₂O: 6.25%; and phenol/MeOH sol: phenol/chloroform/isoamyl alcohol (sigma 77617): 66.7%; MeOH: 33.3%. In some embodiments, chloroform substitute can also be used.

### EXAMPLE

Additional embodiments are disclosed in further detail in the following examples, which are not in any way intended to limit the scope of the claims.

### Example 1

### Recovery of Megabase-Sized DNA from Animal Tissues for Irys™ Mapping

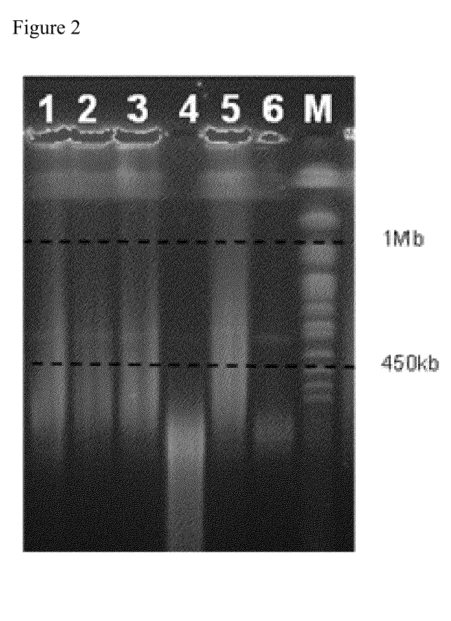

About 50-100 mg of frozen rat liver, brain kidney and 20 mg mouse prostate were homogenized on ice at 10-20 mg/ml MB buffer (10 mM tris, 100 mM NACl, 10 mM EDTA) in a dounce homogenizer. 500 ul aliquots were transferred to microfuge tubes and treated with various concentrations of ethanol and methanol (final concentrations are shown in Table 1) for 45-90 minutes on ice. Following a low speed spin, the resulting cell pellet was immobilized in agarose plugs for subsequent DNA recovery. Results of DNA recovery is shown in Table 2 along with labeling metrics and Center of Mass (COM; size) in Kb. In Table 2, FP=False positive, FN=False negative, Map rate=reference map rate to rat or mouse genome, and COM=average size of DNA in Kb for molecules >20 Kb. Pulse filed gel electrophoresis image showing rat liver DNA treated with alcohol as per this example is shown in FIG. 2.

TABLE 2
Results of Recovery of megabase-sized DNA from animal tissues for irys ™
mapping

| | Rat liver | | | | Rat Brain | | | | Rat Kidney | | | Mouse prostate | | | | |
|---|---|---|---|---|---|---|---|---|---|---|---|---|---|---|---|---|
| Tissue eqivalent | Ethanol | | | | Ethanol | | | Methanol | Ethanol | | Methanol | Ethanol | | | | |
| per plug | 0% | 43% | 55% | 67% | 0% | 55% | 67% | 67% | 55% | 67% | 67% | 0% | 43% | 55% | 67% | |
| 5 mg/10 mg | ND | 5 | 4.9 | 5.3 | 1.1 | 2.9 | 2.1 | 2.4 | 5.2 | 4.6 | 4.6 | ND | 1.8 | 1.9 | 1.6 | DNA |
| tissue | ND | 4.9 | | 4.5 | | | | | | | | | | | | recovery |
| eqivalent per | 4 | 5.1 | 6.4 | 5.6 | 3.3 | 3.8 | 4.3 | 4.1 | 5.3 | 4.1 | 5.9 | ND | | 1.8 | 2.2 | in ug |
| plug | | | | 5.9 | | | | | | | | | | 2.3 | | |
| Irys reference | | 73.9 | 72.9 | 75 | | | | 89.1 | | | | | 66.1 | 69 | 66.4 | |
| map rate(%) | | | | | | | | | | | | | | | | |
| % FP | | 7.1 | 6.9 | 7.3 | | | | 7.5 | | | | | 6.6 | 7.9 | 8.8 | |
| % FP | | 10.5 | 11.3 | 11 | | | | 18.2 | | | | | 16.8 | 12.1 | 11.2 | |
| Label per 100 kb | | 13.1 | 12.8 | 12.9 | | | | 10.9 | | | | | 10.7 | 12.1 | 12.7 | |
| COM (KB) | | 191 | 197.6 | 210.6 | | | | 266 | | | | | 221 | 166.4 | 160 | |

ND = not detectable
DNA recovered without alcohol treatment showed a lower viscosity than alcohol treated samples

### Example 2

### Additional Recovery of Megabase-Sized DNA from Animal Tissues for Irys™ Mapping

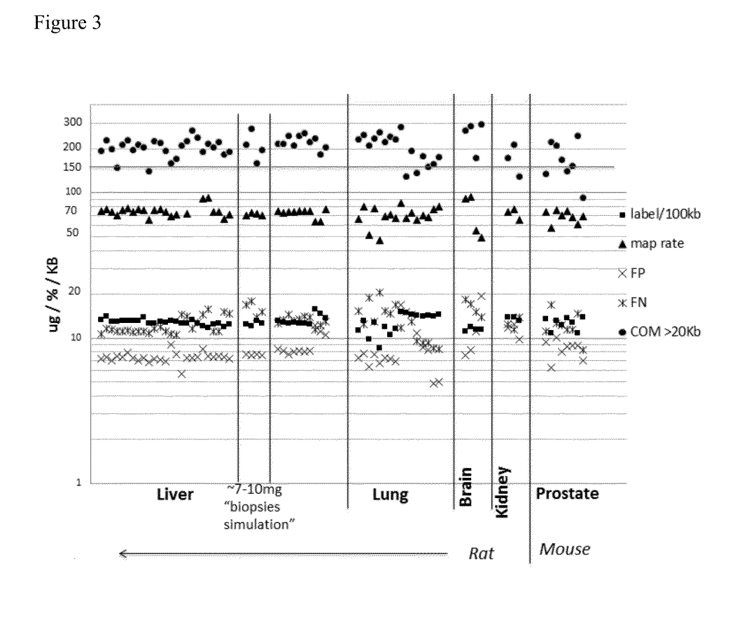

Frozen rat liver (40-100 mg, unless otherwise indicated), lung (40-100 mg), brain (40-100 mg), kidney (40-100 mg) and mouse prostate (about 20 mg) were processed according to the general procedure described in Example 5. DNA was recovered, labeled and run on Irys™ system. The results are shown in FIG. 3, in which labeling metrics (label per 100 kb, Map Rate to reference genome, False Negative (FN), False Positive (FP), and Center of Mass in Kb (COM)) are shown.

### Example 3

### Recovery of Megabase-Sized DNA from Biopsy Amount Rat Lung Tissue for Irys™ Mapping

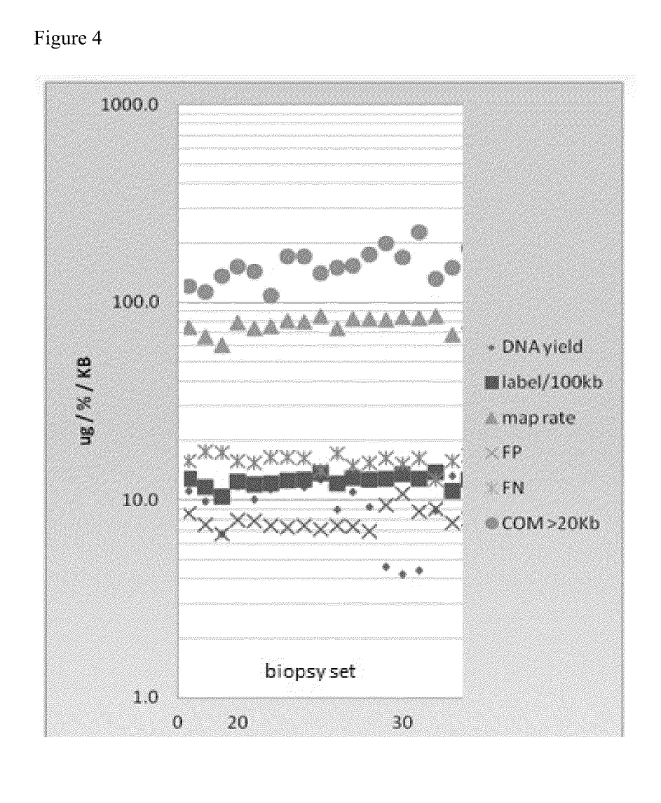

Frozen rat lung tissues (7-10 mg) were processed according to the general procedure described in Example 5. DNA was recovered, labeled and run on Irys™ system. The results are shown in FIG. 4, in which DNA yield in micrograms, labeling metrics (label per 100 kb), Map Rate to reference genome, False Negative (FN), False Positive (FP), and Center of Mass in Kb (COM)) are shown.

### Example 4

### Recovery of Megabase-Sized DNA from Biopsy Amount Rat Lung Tissue for Irys™ Mapping

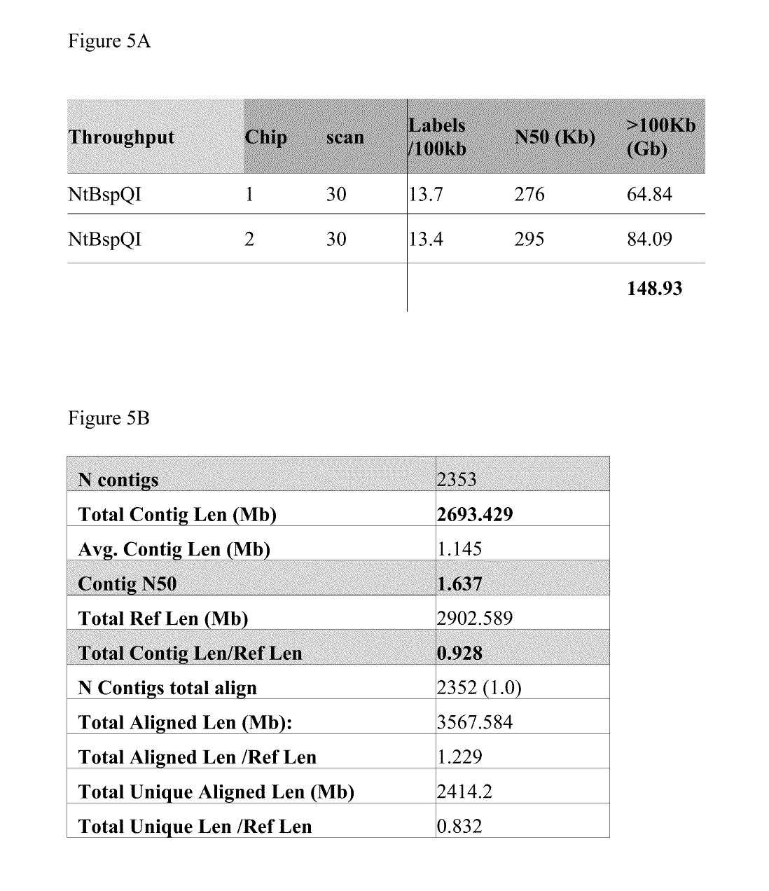

Rat liver DNA was isolated according to the general procedure described in Example 5, labeled and run on Irys™ system. Data was collected and assembled. The label per 100 kb, Center of Mass (N50) in Kb and throughput >100 kb in Gb are depicted in FIG. 4A, and assembly metrics are shown in FIG. 5B. Highlighted in FIG. 5B are the number of contigs (N contig), contig N50 and total contig length to reference length.

### Example 5

### Isolation of Megabase DNA from Animal Tissue

Described in this example is a non-limiting exemplary protocol for processing about 100 mg rat liver in 7 ml Dounce Tissue Grinder for isolation of long DNA molecules from the rat tissue, and should not be used to limit the scope of the present invention. Tissue amount can be scaled up or down pending homogenizer capacity to reflect 1 ml buffer per 20 mg tissue. Larger tissue can be broken under liquid nitrogen using a mortar and pestle and the pieces stored at −80° C. for future use.

For grinding about 100 mg tissue, in 5 ml MB buffer, 7 ml Dounce Tissue Grinder can be used. For grinding up to 20 mg tissue, in about 1 ml MB buffer, 1 ml Tissue Grinder can be used. Frosted pestle mortar combination can facilitate better grinding of tougher tissues

Things to do Before Starting

### Processing Tissue

Place 96-100% ethanol, MB buffer, and Tissue Grinder on ice.

### Embedding in Agarose/Proteinase K Digest

Refer to Plug Lysis Protocol for best practices and critical steps.
Set a heat block or water bath to 70° C. for melting 2% agarose stock.
Set another heat block or water bath to 43° C. for keeping 2% agarose in melted state. (Note: Fill heat block wells with water and equilibrate to the desired temperature just prior to use)
Equilibrate a Thermomixer fitted with 50 ml adapter to 50° C. for Proteinase K digestion.

Protocol Start

Grinding Tissue/Fixing Cells
1. Retrieve frozen tissue from −80° C. storage.
2. Quickly determine weight and transfer to prechilled Tissue Grinder on ice.
3. Quickly add ice-cold MB buffer at 1 ml buffer per 20 mg of tissue.
   Note: addition of 0.15% BME to MB buffer might prove beneficial for some tissues
4. Gently grind by moving pestle up and down about ˜20 times with no twisting while avoiding bubble formation until tissue is disintegrated (tight or loose pestle can be used)
   Note: Check under microscope to ensure efficient tissue pulverization indicative by presence of intact cells. Some components will not disintegrate, i.e. blood vessels. They settle and are avoided in subsequent steps.
5. Incubate homogenate on ice for 5 minutes to settle non-homogenized elements. Aliquot 500 ul of supernatant per microfuge tube avoiding settled material.
6. Add an equal volume ice cold add 96-100% ETOH to each tube.
7. Gently invert 5 times to mix and incubate for 60 minutes on ice, inverting at least once during incubation.
8. Also during incubation, melt 2% agarose at 70° C. for 15 minutes and equilibrate to 43° C. for at least 30 minutes before use. Chill plug casts at 4° C. or on ice for at least 30 minutes.
   Note: chilling plug casts ensures rapid solidification of agarose-sample complex to avoid settling ethanol treated sample to bottom of plugs during solidification process.
9. At the end of 60 minutes ice incubation, spin down cells at 1,500×g for 7 minutes at 4° C.
   Note: pellet forms along the length of the tube up to the solution level.
10. Carefully remove and discard the supernatant with a P1000 tip without disturbing the pellet.

Washing with MB Buffer
11. Resusupend each cell pellet in 1 ml cold MB buffer by first loosening up the pellet with a gentle finger tapping followed by adding 1 ml MB buffer and pipeting up and down with a P1000 tip, being aggressive enough to produce a homogenous suspension.
12. Spin down cells at 1,500×g for 7 min at 4° C.
   Note: centrifugation in MB buffer absent ethanol causes pellet to form at the bottom of the tube.
13. Carefully remove and discard supernatant with a P1000 tip. Remove the last drop with a P200 tip without disturbing the cell pellet.
   a) For 5 mg tissue per plug, resuspend each cell pellet in 125 ul MB buffer.
   b) For 10 mg tissue per plug, resuspend each cell pellet in 66 ul MB buffer.
   Proceed immediately to embed in agarose.
   Note: use 5 mg tissue equivalent per plug for rat liver &kidney, and 10 mg per plug for rat brain.
   Note: If not ready to proceed to next step immediately, cells can be kept on ice; however, all tubes must be brought to room temperature for at least 15 min prior to casting plugs.

Embedding in Agarose Plugs (about 1 Hour)
Fixed cells are equilibrated to room temperature (25° C.) and sequentially mixed, one tube at a time, with 43° C. equilibrate agarose for immediate casting of plugs.
14. Place two P200 pipets near 43° C. water bath or heat block. Set one pipet for casting plugs to the final mix volume as listed in the table below; Set the other P200 pipet to agarose volume, 40 ul or 75 ul.
15. Add 43° C. equilibrated 2% agarose in the volume listed in the table below to achieve a final concentration of 0.75% per plug; Quickly pipet mix the entire volume with casting P200 pipet while avoiding bubble formation and immediately cast plugs (If casting two plugs, withdraw entire 200 ul and fill two plugs sequentially).

# plugs

| | 1 | 2 |
|---|---|---|
| cells in MB buffer | 66 ul | 125 ul |
| 2% agarose | 40 ul | 75 ul |
| total mix volume | 106 ul | 200 ul |

16. Place plug cast on ice for at least 45 minutes to solidify. Alternatively, place plug cast in refrigerator at 4° C. or on inverted metal microfuge block on ice for 45 minutes, to avoid potential contact of agarose with ice.

Proteinase K Digestion (Overnight)

Up to five plugs can be processed simultaneously per 50 ml conical tube. Ensure plugs are fully submerged throughout processing
17. Prepare Proteinase K solution by mixing 200 μl of Qiagen Proteinase K enzyme with 2.5 mls of Lysis Buffer (BioNano Genomics) per 1-5 plugs per 50 ml conical tube.
18. Transfer up to five plugs per conical tube containing Lysis buffer+Proteinase K by first removing tape from bottom of plug cast followed by dislodging with plug mold plunger (Bio-Rad kit). Make sure all plugs are fully submerged in buffer. Use a blunt end spatula for submerging if plugs stick to tube walls.
19. Cap conical tubes and incubate in Thermomixer for 2 hrs at 50° C. with intermittent mixing (mixing cycle: 10 seconds at 450 rpm followed by 10 minutes at 0 rpm).
20. Near the end of the two hours incubation, prepare fresh Proteinase K solution by mixing 200 μl of Qiagen Proteinase K enzyme with 2.5 mls of Lysis Buffer per 1-5 plugs to be processed in each 50 ml conical tube.
21. At end of the two hour incubation, briefly spin 50 ml tubes to collect drops forming on lid. Replace the original cap with the Conical tube sieve (BioRad kit), drain old Proteinase K+Lysis Buffer solution through sieve, and tap bottom of tube on bench surface several times with strong repetitive force to localize plugs at the bottom of conical tube.
22. Remove Conical tube sieve and add freshly mixed Proteinase K solution directly into each 50 ml tube. Account for all plugs and ensure all are submerged in buffer. Tightly recap each tube with its original cap. Incubate overnight at 50° C. with intermittent mixing (mixing cycle: 10 seconds at 450 rpm followed by 10 minutes at 0 rpm).

Day 2: RNase Treatment/Agarose Digestion/Drop Dialysis/DNA Homogeneity Mixing

Things to do Before Starting:
Equilibrate a Thermomixer fitted with 50 ml adapter to 37° C.
Set a heat block or water bath to 43° C. Set another heat block or water bath to 70° C. Note: Fill heat block wells with water and equilibrate to the desired temperature just prior to use. Calibrate temperature with a thermometer.

Protocol Start:

### RNase Treatment

Note: Make sure RNase is DNase free.
1. Briefly spin 50 ml tubes to collect drops forming on lid. Add 50 ul Qiagen RNase Enzyme to lysis buffer proteinase K and incubate at 37° C. for 1 hour with intermittent mixing (mixing cycle: 10 seconds at 450 rpm followed by 10 minutes at 0 rpm).

### Stabilization Wash

2. Prepare 70 mls of 1× Wash Buffer, per 1-5 plugs per 50 ml conical tube, using 10× Wash Buffer (Bio-Rad kit) and molecular biology grade water. Mix thoroughly and store at room temperature until use.
3. At end of the one hour RNase treatment. Replace the original cap with the Conical tube sieve (provided by BioRad kit). Drain solution through sieve and tap tube bottom on bench surface several times with strong repetitive force to localize plugs at bottom of conical tube.
   Note: See Important Notes section of Plug Lysis Protocol for detailed schematics.
4. Rinse plugs three times by: 1) adding 10 mls of 1× Wash Buffer through sieve, 2) swirling tube(s) in a gently for ˜10 seconds, 3) discarding buffer through sieve, and 4) tapping plugs to bottom of conical tube before next rinse. Account for all plugs between rinses and ensure plugs are submerged throughout the rinsing process.
5. Wash plugs four times by: 1) adding 10 mls 1× Wash Buffer through sieve and capping tubes, 2) gently shaking for 15 minutes on horizontal platform mixer at 180 rpm at room temperature, 3) discarding wash solution through the sieve, and 4) tapping plugs to bottom of conical tube before adding the next wash. Account for all plugs between washes and ensure plugs are submerged throughout the washing process. Do not discard the last wash.
   Note: Plugs can be stored in 1× Wash Buffer for up to 2 weeks at 4° C. without significant degradation of DNA quality

DNA Recovery—to recover DNA from plugs, continue with Day 2 of the Plug Lysis Protocol, starting at TE washes.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for isolating DNA from a biological sample, the method comprising:
(a) homogenizing the biological sample to generate a homogenate;
(b) contacting the homogenate with a DNA and/or protein precipitating agent;
(c) embedding the homogenate in a porous matrix; and
(d) recovering DNA from the homogenate.

2. The method of claim 1, wherein cellular integrity, nuclear integrity, or both in the biological sample is at least partially maintained during step (a).

3. The method of claim 1, wherein the DNA and/or protein precipitating agent comprises ethyl alcohol, methyl alcohol, isopropyl alcohol, acetone, or any combination thereof.

4. The method of claim 1, wherein embedding the homogenate in the porous matrix comprises dispersing the homogenate throughout the matrix.

5. The method of claim 1, further comprising contacting the homogenate, following step (b), with a collagenase, an elastase, a lipase, an amylase, a hyaluronidase, an RNase, a fibornectinase, a lamininase, a protease, or any combination thereof.

6. The method of claim 1, wherein the biological sample comprises a plant tissue and wherein homogenizing the biological sample comprises treating the biological sample with a mechanical means, an enzymatic means, or a combination thereof.

7. The method of claim 1, further comprising contacting the homogenate before, after, or during step (b) with a crosslinking agent.

8. The method of claim 6, further comprising contacting the homogenate before, after, or during step (b) with a crosslinking agent.

9. The method of claim 1, further comprising separating one or more discrete DNA-containing entities from tissue fragments, intact cells, and cell remnants before step (c) and after step (b).

10. The method of claim 1, wherein the biological sample comprises a plant tissue, an animal tissue, or both.

11. The method of claim 1, wherein at least 30% of the DNA recovered from the homogenate is more than 20 kilobases.

12. The method of claim 1, wherein recovering DNA from the homogenate comprises treating the porous matrix embedded with the homogenate with an agent to remove non-DNA components.

13. The method of claim 1, wherein recovering DNA from the homogenate comprises contacting the porous matrix embedded with the homogenate with an elastase, a collagenase, hyaluronidase, an RNase, a fibornectinase, a lamininases, a lipase, a carbohydratase, a pectinase, a pectolyase, an amylase, an RNase, a hyaluronidases, or any combination thereof.

14. A method for isolating DNA from a biological sample, the method comprising:
(a) contacting the biological sample with a DNA and/or protein precipitating agent;
(b) homogenizing the biological sample after step (a) to generate a homogenate;
(c) embedding the homogenate in a porous matrix; and
(d) recovering DNA from the homogenate.

15. The method of claim 14, wherein the DNA and/or protein precipitating agent is ethyl alcohol, methyl alcohol, isopropyl alcohol, or acetone.

16. The method of claim 14, further comprising contacting the homogenate, following step (b), with a collagenase, an elastase, a lipase, an amylase, a hyaluronidase, an RNase, a fibornectinase, a lamininase, a protease, or a combination thereof.

17. The method of claim 14, further comprising contacting the homogenate before, after, or during step (a) with a crosslinking agent.

18. The method of claim 14, further comprising contacting the homogenate after, or during step (b) with a DNA and/or protein precipitating agent.

19. The method of claim 14, wherein the biological sample comprises a plant tissue, an animal tissue, or both.

20. The method of claim 14, wherein at least 30% of the DNA recovered from the homogenate is more than 20 kilobases.
